(19) United States
(12) Patent Application Publication
(10) Pub. No.: US 2010/0172831 A1
(43) Pub. Date: Jul. 8, 2010

(54) Protein-Modified Nano-Droplets, Compositions and Methods of Production

(76) Inventors: Thomas G. Mason, Los Angeles, CA (US); Connie B. Chang, Los Angeles, CA (US)

Correspondence Address: VENABLE LLP, P.O. BOX 34385, WASHINGTON, DC, 20043-9998, US

(21) Appl. No.: 12/595,800

(22) PCT Filed: Apr. 18, 2008
(86) PCT No.: PCT/US08/05011

§ 371 Date: Oct. 13, 2009

Related U.S. Application Data
(60) Provisional application No. 60/907,824, filed on Apr. 18, 2007.

(51) Int. Cl. A61K 9/51; A61K 51/00

(52) U.S. Cl. 424/1.29; 424/491; 977/773; 977/906

(57) ABSTRACT

A protein-modified droplet includes a droplet having a liquid material, and a protein structure formed to at least partially enclose the droplet. The protein structure includes a plurality of protein molecules having an affinity to at least a region of the droplet during formation of the protein structure, and the droplet has a maximum dimension of at least about 1 nm and less than about 1000 nm. A composition includes a plurality of protein-modified droplets dispersed in an aqueous solution.

### CROSS-REFERENCE TO RELATED APPLICATION

This application claims priority to U.S. Provisional Application No. 60/907,824 filed Apr. 18, 2007, the entire contents of which are hereby incorporated by reference.

### BACKGROUND

1. Field of Invention This application relates to nanodroplets, and more particularly to protein-modified nanodroplets and compositions, and methods of production.

2. Discussion of Related Art

The contents of all references, including articles, published patent applications and patents referred to anywhere in this specification are hereby incorporated by reference.

Pure viral capsid protein can be self assembled around nanoscale objects, (Bancroft, J. B.; Hiebert, E. Formation of an Infectious Nucleoprotein from Protein and Nucleic Acid Isolated from a Small Spherical Virus, Virology 1967, 32, 354-356; Bancroft, J. B.; Hills, G. J.; Markham, R. A Study of the Self-Assembly Process in a Small Spherical Virus. Formation of Organized Structures from Protein Subunits in Vitro. Virology 1967, 31, 354-379; Hiebert, E.; Bancroft, J. B.; Bracker, C. E. The Assembly in Vitro of Some Small Spherical Viruses, Hybrid Viruses, and Other Nucleoproteins, Virology 1968, 34, 492-508) enclosing them in protein shells through a process known as “encapsidation”. (Douglas, T.; Strable, E.; Willits, D.; Aitouchen, A.; Libera, M.; Young, M. Protein Engineering of a Viral Cage for Constrained Nanomaterials Synthesis, Adv. Mater. 2002, 14, 415-418; Douglas, T.; Young, M. Host-Guest Encapsulation of Materials by Assembled Virus Protein Cages, Nature 1998, 393, 152-155; Douglas, T.; Young, M. Virus Particles as Templates for Materials Synthesis, Adv. Mater. 1999, 11, 679-681; Dragnea, B.; Chen, C.; Kwak, E. S.; Stein, B.; Kao, C. C. Gold Nanoparticles as Spectroscopic Enhancers for in Vitro Studies on Single Viruses, J. Am. Chem. Soc. 2003, 125, 6374-6375.)

By displaying viral protein, encapsidated nanomaterials can potentially be endowed with a desirable viral functionality: preferential localization in specific tissues that could be useful for cell targeting (Uchida, M.; Klem, M. T.; Allen, M.; Suci, P.; Flenniken, M.; Gillitzer, E.; Varpness, Z.; Liepold, L. O.; Young, M.; Douglas, T. Biological Containers: Protein Cages as Multifunctional Nanoplatforms, Adv. Mater. 2007, 19, 1025-1042). In a classic demonstration of encapsidation, an infectious virus was assembled in vitro by combining pure capsid protein with pure RNA and dialyzing to change pH and ionic strength (Bancroft, J. B.; Hiebert, E. Virology 1967, 32, 354-356). Likewise, synthetic polymers (Bancroft, J. B.; Hiebert, E.; Bracker, C. E. The Effects of Various Polyanions on Shell Formation of Some Spherical Viruses, Virology 1969, 39, 924-930), parapolyoxometalate particles (Douglas, T.; Young, M. Host-Guest Encapsulation of Materials by Assembled Virus Protein Cages, Nature 1998, 393, 152-155), solid gold nanocrystals (Dragnea, B.; Chen, C.; Kwak, E. S.; Stein, B.; Kao, C. C. Gold Nanoparticles as Spectroscopic Enhancers for in Vitro Studies on Single Viruses, J. Am. Chem. Soc. 2003, 125, 6374-6375; Chen, C.; Daniel, M. C.; Quinkert, Z. T.; De, M.; Stein, B.; Bowman, V. D.; Chipman, P. R.; Rotello, V. M.; Kao, C. C.; Dragnea, B. Nanoparticle-Templated Assembly of Viral Protein Cages, Nano Lett. 2006, 6, 611-615; Sun, J.; DuFort, C.; Daniel, M.-C.; Murali, A.; Chen, C.; Gopinath, K.; Stein, B.; De, M.; Rotello, V. M.; Holzenburg, A.; et al. Core-Controlled Polymorphism in Virus-Like Particles, Proc. Natl. Acad. Sci. U.S.A. 2007, 104, 1354-1359), and quantum dots (Dixit, S. K.; Goicochea, N. L.; Daniel, M.-C.; Murali, A.; Bronstein, L.; De, M.; Stein, B.; Rotello, V. M.; Kao, C. C.; Dragnea, B. Quantum Dot Encapsulation in Viral Capsids, Nano Lett. 2006, 6, 1993-1999) have been encapsidated to create virus-like particles (VLPs) similar in size to the native virus. For such small VLPs, electron microscopy indicates that the protein shell assembles from individual subunits in a manner reminiscent of micelle formation (McPherson, A. Micelle Formation and Crystallization as Paradigms for Virus Assembly, BioEssays 2005, 27, 447-458) into ordered structures characteristic of icosahedral viruses (Zandi, R.; Reguera, D.; Bruinsma, R. F.; Gelbart, W. M.; Rudnick, J. Origin of Icosahedral Symmetry in Viruses, Proc. Natl. Acad. Sci. U.S.A. 2004, 101, 15556-15560), including protruding ring-like multimers, or “capsomers”, that have five-fold and six-fold symmetry (Caspar, D. L.; Klug, A. Physical Principles in the Construction of Regular Viruses, Cold Spring Harb. Symp. Quant. Biol. 1962, 27, 1-24). However, the prior art encapsidation materials and techniques have been of limited utility to date. There thus remains a need for improvements.

### SUMMARY

A protein-modified droplet according to an embodiment of the current invention includes a droplet comprising a liquid material, and a protein structure formed to at least partially enclose the droplet. The protein structure comprises a plurality of protein molecules having an affinity to at least a region of the droplet during formation of the protein structure, and the droplet has a maximum dimension of at least about 1 nm and less than about 1000 nm. A composition according to an embodiment of the current invention comprises a plurality of protein-modified droplets according an embodiment of the current invention dispersed in an aqueous solution.

A method of producing protein-modified droplets according to an embodiment of the current invention includes supplying first and second immiscible liquid materials; adding a stabilizing agent to at least one of the first and second immiscible liquid materials; emulsifying the first and second liquid materials to form a plurality of droplets of the second liquid material in the first liquid material that are stabilized by the stabilizing agent, each droplet of the plurality of droplets having a maximum dimension of at least about 1 nm and less than about 100 nm; adding protein molecules at least one of prior to or after said emulsifying; and allowing a protein structure to form to at least partially enclose each of the plurality of droplets. The stabilizing agent and the protein molecules added are of types that have mutual electrostatic attractions to each other when the stabilizing agent is attached to the droplets.

### BRIEF DESCRIPTION OF THE DRAWINGS

The invention is better understood by reading the following detailed description with reference to the accompanying figures in which:

FIG. 1 is a schematic illustration showing the encapsidation of an oil droplet stabilized by anionic sodium dodecyl sulfate (SDS) surfactant in water by purified capsid protein from cowpea chlorotic mottle virus (CCMV) according to an embodiment of the current invention. This is one example of a protein-modified droplet. By adjusting the pH and ionic strength I using dialysis, the capsid protein in bulk solution can be induced to condense and assemble around the negatively charged surfaces of the nanoemulsion droplets.

FIGS. 2(a) and 2(b) show capsid protein structures observed by negatively stained TEM according to an embodiment of the current invention. FIG. 2(a) shows individual nanoscale droplets as a function of pH and ionic strength/of NaCl after mixing and dialyzing SDS-stabilized nanoemulsions with purified CCMV protein. Buffers are: RNA-reassembly (R) (pH=7.2, I=0.1 M); hexagonal sheet (H) (pH=6.2, I=0.1 M); dimer (D) (pH=6.2, I=1.0 M); multi-shell (M) (pH=4.8, I=0.1 M); and empty shell (E) (pH=4.8, I=1.0 M). Inset (upper right): Fluorescence optical micrograph of FITC-labeled CCMV protein (green) covering the surfaces of microscale silicone oil droplets stabilized by SDS after dialysis with R buffer. FIG. 2(b) shows nanodroplets encapsidated by 1, 2, and 3 concentric protein shells are observed after dialysis with M buffer. Scale bar=20 nm (all images).

FIG. 3 shows representative examples of CCMV protein structures observed as a function of the droplet diameter, d (italic numbers), on a single side of individual encapsidated oil nanodroplets after dialysis using RNA-reassembly buffer according to an embodiment of the current invention. TEM images have been background subtracted and Fourier filtered to enhance the protein structures on the droplet surfaces. Complete protein ‘capsomers’ (white rings) are found more often on the surfaces of smaller nanodroplets that have sizes closer to that of the native virus. Ring-like capsomers can order into six-fold arrangements locally (dark circle). Extended dark trough-like ‘scars’ (dark circle), defected capsomers, and hexagonal web-like networks of capsid protein (dark circle) are more frequently seen on larger droplets. Allowed triangulation numbers T and predicted outer diameters of nanodroplets that could be encapsidated by perfect icosahedra of ordered capsomers are shown in the lower scale. The outer diameters (in nm) are estimated using: d(T)≈28(T/3)^{1/2}, consistent with d=28 nm for CCMV, a T=3 virus.

FIGS. 4(a)-4(c) show local protein structures observed on the surfaces of nanodroplets (enlarged from dark circles in FIG. 3) have different degrees of order and disorder. FIG. 4(a) shows six-fold coordinated capsomers (dots at center) represent a high degree of order seen mostly on smaller droplets (left side). An example of a trough-like scar that consists of an elongated dark region (arrow) surrounded by a protruding white region (middle). Hexagonal web structure, typically seen on larger droplets, consists of dark spots (dots) surrounded by an interconnected white network of protein protruding from the interface (right side). FIG. 4(b) shows probabilities p_{c} and p_{w} versus distance, r, between centers of dark regions for hexagonal capsomers and web, respectively. The average spacing between the dark spots of the web (4.7 nm) is roughly half of the distance between the centers of capsomers (9.5 nm). FIG. 4(c) shows a web-like structure (right side) can be made by packing hexagonal capsomers (lower left) of hand-in-glove protein dimers (upper left) on a flat surface. Regions of low protein density are marked in one hexagonal cell with black dots.

### DETAILED DESCRIPTION

In describing embodiments of the present invention illustrated in the drawings, specific terminology is employed for the sake of clarity. However, the invention is not intended to be limited to the specific terminology so selected. It is to be understood that each specific element includes all technical equivalents which operate in a similar manner to accomplish a similar purpose.

According to some embodiments of the current invention, we provide a process for creating nanoemulsion droplets modified by and/or covered by protein. In some embodiments, the protein can effectively provide a capsule or container which can be loaded with selected materials. Such containers can provide a drug delivery structure in some embodiments of the current invention. However, the broad concepts of the invention are not limited to only drug delivery. In addition, a protein capsule containing a liquid droplet therein is only one example of a protein-modified droplet according to an embodiment of the current invention. For instance, a protein capsule could contain a nanoporous polymeric gel particle that is loaded with selected materials.

In native viruses, the viral coat protein of the virus serves as a barrier to protect its interior contents, the nucleic acid RNA or DNA, which is necessary for self-propagation and genomic reproduction. Viruses have the ability to readily penetrate specific cells, so some embodiments of the current invention may include targeting delivery of particular drugs to certain cells by tailoring the type of viral coating on the surface of the droplets. Thus, some embodiments of the current invention can provide a capsule that mimics some aspects of the natural virus. This may include, in some embodiments, providing a capsule that can penetrate cell barriers and deliver the contents inside the cell.

In one embodiment, we obtained viral capsid protein through a standard method of growing the virus, disassembling it, and separating the protein from the genetic material (RNA or DNA). However, the broader concepts of the invention are not limited to only such techniques and those particular proteins. In an alternative, the capsid protein can be obtained in larger quantities through bacterial expression of the viral RNA. Next, we prepared microscale emulsions or nanoscale emulsions (nanoemulsions) of hydrophobic oil in water. The hydrophobic drug molecules readily dissolve in the oil, yet the oil is not so low in molecular weight that the emulsion destabilizes through Ostwald ripening. The concentration of the drug molecules is fixed in the oil, and then the drug-laden oil is used as a feed for the next step, i.e., the production of oil-in-water emulsions through shear emulsification. The extreme emulsification process used to make nanoemulsions in one example involved using a commercial high-pressure microfluidic device. An ultrasonic device and other methods can also be used in accordance with the invention.

Droplets comprised of liquid can be encapsulated with viral proteins, yielding a dispersion of viral protein-coated droplets of one liquid in a different immiscible liquid through several different methods according the various embodiments of the current invention. Some methods according the current invention include the following: (1) adding oil of the desired type to an aqueous dispersion of viral capsid protein while controlling the droplet stabilization through type and concentration of stabilizing agents (e.g. surfactants, particles, or polymers) and also controlling the pH, ionic content (e.g. types of salts or buffers), and ionic strength (e.g. concentrations of salts or buffers) and applying a mechanical shear or otherwise inducing a flow that can cause bigger droplets to break down into smaller droplets; (2) combining an existing oil-in-water emulsion or nanoemulsion (stabilized by charge surfactant, particles, or polymers) to an aqueous dispersion of viral capsid protein at an appropriate pH, ionic content, and ionic strength and mixing in a manner that does not cause droplet break-up but does distribute the components by convection; and (3) combining an existing oil-in-water emulsion or nanoemulsion with an aqueous dispersion of viral capsid protein and then dialyzing using a semi-permeable membrane to change the pH, ionic content, and ionic strength in order to cause the adsorption of the protein onto the surfaces of the droplets.

The liquid material of the droplets can include one or more of the following materials: an oil, a silicone oil, a hydrocarbon oil, a petroleum oil, a fuel oil, a wax, a fat, a fluorinated oil, a non-volatile oil, a volatile oil, an aromatic oil, an oil derived from a plant material, an oil derived from an animal material, an oil derived from a natural source, a distilled oil, an extracted oil, a cooking oil, a food oil, a lubricant, a reactive material that is predominantly hydrocarbon in composition, an epoxy material, an adhesive material, a polymerizable material, a thermotropic liquid crystal, a lyotropic liquid crystal, an acidic oil, a basic oil, a neutral oil, a natural oil, a polymer oil, and a synthetic oil.

Biologically active agents according to some embodiments of the current invention can include, but are not limited to, drug molecules, anti-cancer molecules, therapeutic molecules, hormone molecules, agonist molecules, antagonist molecules, inhibitor molecules, suppressor molecules, sensitizer molecules, antidepressant molecules, antiviral molecules, antifungal molecules, antibacterial molecules, bioavailability enhancer molecules, toxin molecules, dye molecules, fluorescent molecules, biomolecules, nutrients, vitamins, flavors, enzymes, nanoparticles, and imaging contrast enhancement agents.

A surfactant, such as negatively charged sodium dodecyl sulfate (SDS) can be added to give the emulsion droplets stability against subsequent coalescence after they are created through flow-induced rupturing of bigger droplets into smaller droplets. Alternatively, commercial mixers, blenders, colloid mills, or flow-focusing microfluidic devices could be used to create the emulsions or nanoemulsions out of the oil containing the drug molecules. Existing methods of extreme flow are capable of creating droplets down to about 5-10 nm in radius, so that only a very small number of drug molecules may be in a given droplet. These smaller nanodroplets themselves can penetrate cellular and intestinal membranes more readily through enhanced diffusion and penetration of pores, and the viral coating gives them a sturdiness and active means of traversing membranes through protein triggering of cellular uptake. Since the droplets can be produced in large quantities in some embodiments of the current invention, the viral protein often being a limiting ingredient, we typically do not emulsify with the protein present, although this can be done in some embodiments of the current invention. Instead, we obtain the droplets, dilute them and fix the surfactant concentration, and then add the disassembled viral capsid protein in an embodiment of the current invention. By then changing the ionic strength of the solution and/or the pH, we can cause the protein to become attracted to the droplet surfaces and assemble a coating on the droplets. In some embodiments, we use an anionic surfactant to stabilize the droplets, and this causes the droplets to have a negative charge on their surfaces. This mimics RNA and DNA, which are also negatively charged in solution. Then, we add disassembled capsid protein and change the ionic strength and pH of the solution to cause the viral shell to form on the surfaces of the droplets. To demonstrate this principle, we have performed the first viral encapsulation experiments of nanodroplets using silicone nanoemulsions coated with an anionic surfactant, sodium dodecylsulfate (SDS), and capsid protein obtained from cowpea chlorotic mottle virus (CCMV), a plant virus. No specific drug molecules were added to the oil in that example. In other examples, we have added other oil-soluble molecules into our nanodroplets, such as fluorescent dyes. Our transmission electron micrograph images show the successful assembly of the viral protein on the surfaces of the droplets. One can optimize pH and ionic strength to fully coat the droplets without causing empty virus shells to form. These empty shells waste the protein, so they are not typically desirable. Under certain conditions of composition and assembly, we have also observed that several inner droplets can be enclosed within a single outer shell of protein that forms around them. Overall, we describe methods that can be used to create emulsion and nanoemulsion droplets of a very wide range of sizes that are coated by the viral protein and can have enhanced ability to trigger rapid penetration, targeting, and delivery. By controlling the size distribution of the droplets in some embodiments, one can control the release of the drug since larger droplets will penetrate more slowly than smaller droplets. Alternatively, other proteins synthesized or purified by known methods could be used to coat the droplets.

### Example 1

In an example according to one embodiment of the current invention, we use the capsid protein from the CCMV (Cowpea Chlorotic Mottle Virus), which self-assembles at the surface of nanoemulsion droplets due to electrostatic interactions. In the native virus, the positively-charged interior of the virus interacts with one or more negatively-charged polyanions of RNA. Since the nanoemulsion droplets have negatively-charged surfactant head groups on the exterior of the droplets, the viral proteins assemble at the exterior interface of the oil droplet.

### Procedure for Obtaining Capsid Protein:

We adopt Rao's procedure for purification of the CCMV protein (Choi, Y. G.; Rao, A. L. N., Molecular Studies on Bromovirus Capsid Protein: VII. Selective Packaging of BMV RNA4 by Specific n-Terminal Arginine Residues. Virology 2000, 275, 207-217). We start first with wild-type CCMV at a concentration of 4 mg/mL in suspension buffer. The CCMV is dialyzed in disassembly buffer for 24 hours in order to dissociate the CCMV into protein dimers and RNA. The disassembled CCMV is removed from the buffer and centrifuged for 30 minutes at 14,000 rpm (Eppendorf Centrifuge 580 4R) to precipitate the RNA. The protein in the supernatant is extracted and then further dialyzed in RNA assembly buffer for 24 hours in order to assemble around RNA left in the supernatant. Finally, the supernatant is centrifuged for 1:40 hours at 100,000 rpm (Beckman TLA 110 UC) and the upper ¾ of the supernatant, which contains the pure CCMV protein, is used for further study. The purity and concentration of the resultant protein is measured using UV-visible spectroscopy. All work is done at and 4° C.

### Procedure for Making Nanoemulsion Droplets:

Nanoemulsions, droplets of one liquid phase stabilized in another immiscible liquid phase by surfactant, with diameters less than 100 nm, were created using extreme shear with a microfluidic injection system. The size of the nanoemulsion droplets is dependent upon the amount and type of surfactant used, the pressures at which the liquids are injected into the microfluidic system, and the viscosities of the liquids. The nanoemulsions were then centrifuged and fractionated in order to obtain a specific size distribution of the droplets (Mason, T. G., J. N. Wilking, K. K. Meleson, C. B. Chang, and S. M. Graves. 2006. Nanoemulsions: formation, structure, and physical properties, Journal of Physics: Condensed Matter 18: R635-R666; Meleson, K., S. Graves, and T. Mason. 2004. Formation of Concentrated Nanoemulsions by Extreme Shear. Soft Materials 2: 109-123). We typically make oil-in-water nanoemulsion droplets, the size of which can be controlled through the microfluidic device and other compositional parameters. Thus, this embodiment is for packaging hydrophobic drugs inside a droplet that is in turn inside a viral capsid shell.

Assembly Conditions (Combining Viral Proteins with Nanoemulsion Droplets):

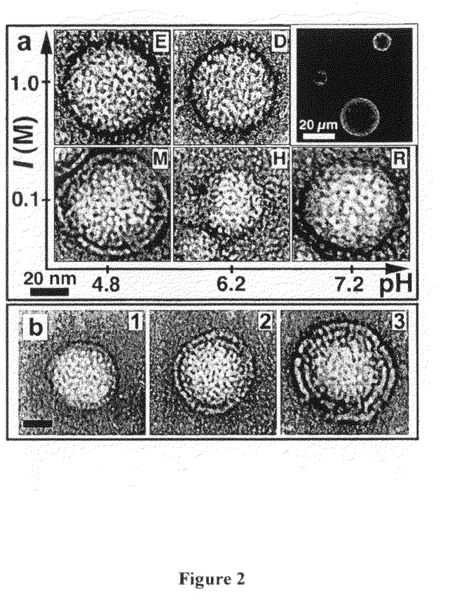

We have used various assembly conditions to assemble viral proteins around nanoemulsion droplets. By varying pH and ionic strength of the solutions against which the nanoemulsion droplets and viral proteins are dialyzed, one can create droplets that have a single coat of viral protein on the outside, a double coat, or multiple coats (see FIG. 2(b)).

### Procedure for Taking EM Images:

Copper grids of 400-mesh size (Ted Pella Inc., Redding, Calif.) were prepared using support films of parlodoin, and then carbon-coated. The grids are glow-discharged by using high-voltage, alternating current, immediately before sample deposition. Sample deposition steps consisted of placing 5 pL of the sample directly on to the grid for 1 minute, wicking with Whatman 4 filter paper, immediately staining with 1% uranyl acetate for 1 minute, wicking again, and air-drying. Samples were viewed under a Hitachi H-7000 electron microscope at an accelerating voltage of 75 kV. Negatives were developed and scanned using a Minolta Dimage Scan MultiPro scanner for image analysis.

### Discussion of Results:

Advantages of this method for producing droplets covered by viral protein according to some embodiments of the current invention can include the ability to fine-tune the size of the nanoemulsion, which is the template for viral assembly. Thus, we are able to vary the diameter of this protein container from about 10 nm to 100 nm, for example, below 1/10 of a micron, allowing size-specific variants for future applications. The adsorption of the viral capsid protein onto the surfaces of the droplets can be controlled by the affinity of the protein for the oil and surfactant on the surfaces of the droplets, not by the droplet size. Therefore, it is possible for us to also make sub-micron, microscale, and even larger virally encapsulated droplets, if these would be desired.

Some embodiments of this invention can provide methods to produce protein-modified droplets for delivering biologically active contents (hydrophobic drug) into the interior of an organism through ingestion, injection, inhalation, or through the skin. Molecules that contain radioactive species or high atomic number elements could be inserted into the nanodroplets for cancer treatment or imaging enhancement. Thus, some embodiments of this invention could have potential applications in both medical imaging and drug delivery. In medical imaging, one application can be the use of the container in tracing pathways of transport within the cell. In drug delivery, one application can be the use of therapeutic agents encapsulated in the nanoemulsion and subsequently delivered upon entry of cancerous cell to treat cancer.

### Example 2

This example is the encapsidation of incompressible spherical nanodroplets, or ‘nanoemulsions’, that can have a continuous range of sizes extending significantly beyond the wild-type core and are stabilized by adsorbed anionic surfactant molecules. We show that it is possible to force the capsid protein to self-assemble into spherical shells without the perfect symmetry and discrete sizes of ideal icosahedra dictated by the Caspar-Klug hierarchy (Caspar, D. L.; Klug, A., Physical Principles in the Construction of Regular Viruses. Cold Spring Harb. Symp. Quant. Biol. 1962, 27, 1-24), which requires special integral multiples (e.g. 1, 3, 4, 7, . . . ) of 60 proteins. Silicone oil (poly-dimethylsiloxane)-in-water nanoemulsions stabilized by sodium dodecyl sulfate (SDS) are made by high-pressure homogenization (Meleson, K.; Graves, S.; Mason, T. G., Formation of Concentrated Nanoemulsions by Extreme Shear. Soft Materials 2004, 2, 109-123), mixed with pure cowpea chlorotic mottle virus (CCMV) capsid protein (Choi, Y. G.; Rao, A. L. N., Molecular Studies on Bromovirus Capsid Protein: VII. Selective Packaging of BMV RNA4 by Specific n-Terminal Arginine Residues. Virology 2000, 275, 207-217), and dialyzed to reduce the divalent cation concentration, causing the protein to self-assemble (Adolph, K. W.; Butler, P. J. G., Reassembly of a Spherical Virus in Mild Conditions. Nature 1975, 255, 737-738). Over a wide range of pH and ionic strength, the re-assembly creates virus-like droplets (VLDs) coated by a single protein shell. We also explore a broad range of pH and ionic strength to control the number of concentric shells formed by the capsid protein around the nanodroplets. In the limit of low pH and ionic strength, where empty multi-shell structures have been formed (Adolph, K. W.; Butler, P. J., Studies on the Assembly of a Spherical Plant Virus. I. States of Aggregation of the Isolated Protein. J. Mol. Biol. 1974, 88, 327-341), droplets can be encapsidated inside two or more protein shells.

For VLDs coated by single shells, transmission electron microscopy (TEM) reveals that the protein has self-assembled on the curved surfaces not only into ordered capsomers but also into a variety of other structures. As the droplet surface curvature is reduced, ordered capsomer structures become less prevalent, and other protein structures appear: defected capsomers, hexagonal webs, and trough-like scars. Some of these structures appear to be due to jamming (Liu, A. J.; Nagel, S. R., Jamming Is Not Just Cool Any More. Nature 1998, 396, 21-22) of the protein on the curved surface and are reminiscent of defects found on macroscopic droplets stabilized by solid microscopic particles (Bausch, A. R.; Bowick, M. J.; Cacciuto, A.; Dinsmore, A. D.; Hsu, M. F.; Nelson, D. R.; Nikolaides, M. G.; Travesset, A.; Weitz, D. A., Grain Boundary Scars and Spherical Crystallography. Science 2003, 299, 1716-1718; Bowick, M.; Cacciuto, A.; Nelson, D. R.; Travesset, A., Crystalline Order on a Sphere and the Generalized Thomson Problem. Phys. Rev. Lett. 2002, 89, Art. No. 185502 pp. 1-4; Tarimala, S.; Dai, L. L., Structure of Microparticles in Solid-Stabilized Emulsions. Langmuir 2004, 20, 3492-3494). However, other structures, such as the hexagonal web, arise from special rules associated with attractive protein-protein and protein-surface interactions. The overall reduction in the population of ordered capsomers on larger droplets implies that the three different conformations of the protein (Speir, J. A.; Munshi, S.; Wang, G.; Baker, T. S.; Johnson, J. E., Structures of the Native and Swollen Forms of Cowpea Chlorotic Mottle Virus Determined by X-Ray Crystallography and Cryo-Electron Microscopy. Structure 1995, 3, 63-78) in the capsid of CCMV are not present in the same proportions when the protein assembles on incompressible surfaces that have lower curvature. Thus, surface curvature plays an important role in setting protein conformation and profoundly influences the structure of assembled proteins that encapsidate incompressible objects.

### Methods

### Protein Purification

Following Choi and Rao's procedure (Choi, Y. G.; Rao, A. L. N., Molecular Studies on Bromovirus Capsid Protein: VII. Selective Packaging of BMV RNA4 by Specific n-Terminal Arginine Residues), we isolate and purify capsid protein from CCMV. CCMV has a single capsid protein, so any reference to ‘CCMV protein’ therefore specifies CCMV's single unique capsid protein. Purified CCMV is dialyzed for 24 hours in 1.0 L of disassembly buffer (0.5 M CaCl₂, 50 mM Tris-HCl at pH 7.5, 1.0 mM EDTA, 1.0 mM DTT, 0.5 mM PMSF). The dissociated virus is centrifuged for 30 minutes at 14,000 RPM to precipitate the RNA. The protein supernatant is extracted and dialyzed for 24 hours in 1.0 L of RNA reassembly buffer (50 mM NaCl, 50 mM Tris-HCl, pH 7.2, 10 mM KCl, 5.0 mM MgCl₂, 1.0 mM DTT). The solution is then centrifuged for 100 minutes at 100,000 RPM, and the protein supernatant is extracted. The concentration and purity of the protein have been measured using UV-visible spectroscopy. All work has been performed at 4° C.

### Nanoemulsion Preparation and Fractionation

Nanoemulsions are created using extreme flow with a high-pressure microfluidic device (Meleson, K.; Graves, S.; Mason, T. G., Formation of Concentrated Nanoemulsions by Extreme Shear. Soft Materials 2004, 2, 109-123). Polydisperse emulsions are size-fractionated using ultracentrifugation to achieve better droplet uniformity and to set the SDS concentration C_{SDS}. Prior to mixing with protein and dialyzing, the nanoemulsions have C_{SDS}=1 mM SDS, well below the critical micelle concentration, and φ=0.05. The PDMS oil (10 cSt viscosity, supplied by Gelest) has a low vapor pressure, so it does not evaporate over the time scale of these microscopy measurements, even when capsid protein is not present.

### Dialysis Buffers

RNA reassembly buffer (Adolph, K. W.; Butler, P. J., Assembly of a Spherical Plant Virus. Philos. Trans. R. Soc. Lond. B 1976, 276, 113-122): Tris-HCl buffer at pH=7.2, I=0.10 M NaCl, 10 mM KCl, 5.0 mM MgCl₂, and 1.0 mM DTT. Empty shell buffer: 50 mM sodium acetate buffer at pH=4.8 and I=1.0 M NaCl. Dimer buffer: 50 mM of sodium phosphate buffer at pH=6.2 and I=1.0 M NaCl. Multi-shell buffer: 50 mM sodium acetate buffer at pH=4.8 and I=0.1 M NaCl. Hexagonal sheet buffer: 50 mM sodium phosphate buffer at pH=6.2 and I=0.1 M NaCl. The last four buffers also contain 1.0 mM EDTA and 1.0 mM DTT.

### Encapsidation Procedure

A 10 μL aliquot of stock nanoemulsion at 1.0 mM SDS and φ=0.05 is added to purified CCMV protein at 0.15 μg/mL to give a total reaction volume of 200 μL. The mixture is dialyzed in 1.0 L of the appropriate buffer for 24 hours at 4° C. The SDS concentration after dilution and dialysis is roughly 10⁻⁵ M, so binding of SDS-protein interaction in the bulk solution is minimized while still maintaining droplet stability. The sulfate head-group of SDS remains negatively charged over the entire range of pH we access. After dilution, the charge density of SDS on the oil-water interfaces is estimated to be roughly −0.1 e/nm².

### Transmission Electron Microscopy: Staining and Analysis

Pelco copper grids of 400 mesh size and 3.0 mm OD (Ted Pella, Inc.) are coated with a thin film of parlodion and carbon. The grids are glow-discharged using high-voltage, alternating current, immediately before sample deposition. We place 5 μL of the sample directly onto the grid for 1 minute, then wick with Whatman 4 filter paper, and immediately stain with a 1% solution of uranyl acetate in water for 1 minute. The samples are air-dried and viewed under a Hitachi H-7000 electron microscope at an accelerating voltage of 75 kV. Negatives were developed and scanned using a Minolta Dimage Scan MultiPro scanner for image analysis. Adobe Photoshop is used to flatten the image background by subtracting a strongly blurred image. Cross-correlation Fourier-transform image analysis is applied using a correlation kernel that has a dark center, corresponding to the size of the capsomer's dark dimple and a white outer ring.

### Fluorescence Microscopy

We have made stock solutions of FITC and 5(6)-FAM, SE in DMSO at 1.0 mg/mL. An aliquot of the 5(6)-FAM, SE stock is added to the dissociated CCMV protein in RNA reassembly buffer at pH 7.2. Another aliquot of the FITC stock solution is added to dissociated CCMV protein and equilibrated in 50 mM phosphate buffer at a pH of 8.2. The protein and dye are mixed, and, after 8 hours, the FITC-labeled protein is dialyzed into RNA reassembly buffer, lowering the pH. Both sets of the fluorescently-labeled protein are mixed with 10 μL of microscale emulsions at 1.0 mM concentrations of either SDS (or CTAB) and φ=0.05 in a total reaction volume of 200 μL and dialyzed using RNA reassembly buffer. Fluorescence micrographs reveal the presence of labeled protein at the droplet surfaces through strong fluorescence at the edges of the droplets. Microscale emulsions in the absence of labeled protein do not show this fluorescence. Therefore, droplets that are much larger than the native virus can be coated by a dual-layer consisting of a first inner layer of anionic surfactant and a second outer layer of virus protein. The adsorption of the protein likely inhibits the equilibrium exchange of surfactant to and from the droplet interfaces. This protein adsorption is typically irreversible for neutral and acidic conditions of pH over a wide range of ionic strength in the solution surrounding the protein-modified droplet. After assembly, the protein-modified droplet can be disassembled by causing the solution conditions to enter a region that would cause the disassembly of native virions.

### Results and Discussion

Anionically stabilized nanodroplets provide incompressible, charged templates that offer a wide range of curvatures upon which capsid protein can be assembled. Through extreme emulsification, we make oil-in-water nanoemulsions comprised of spherical droplets that can be as small as CCMV (inner diameter of 21 nm and outer diameter of 28 nm) (Mason, T. G.; Wilking, J. N.; K. Meleson, K.; Chang, C. B.; Graves, S. M., Nanoemulsions: Formation, Structure, and Physical Properties. J. Phys.: Condens. Matter 2006, 18, R635-R666). Relying upon differences in creaming rates, ultracentrifugal size-fractionation provides uniform model nanoemulsions having droplet radii between 10 nm<a<100 nm (Meleson, K.; Graves, S.; Mason, T. G., Formation of Concentrated Nanoemulsions by Extreme Shear. Soft Materials 2004, 2, 109-123). In addition, the droplet volume fraction φ and surfactant concentration C_{SDS} can be set independently. The Laplace pressure, corresponding to the stress necessary to overcome surface tension and deform a droplet, is typically above 10 atm, so droplets are spherical at dilute φ. To inhibit Ostwald ripening (Taylor, P., Ostwald Ripening in Emulsions: Estimation of Solution Thermodynamics of the Disperse Phase. Adv. Colloid Interface Sci. 2003, 106, 261-285), which can lead to unwanted growth of the droplets through molecular diffusion, the dispersed liquid is chosen to be very insoluble in the continuous liquid phase.

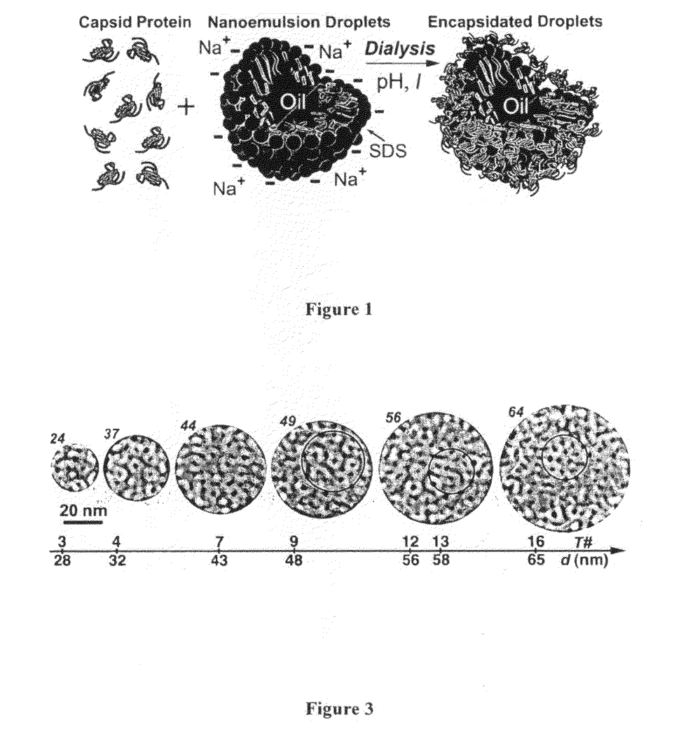

We create virus-like droplets by mixing pure, disassembled CCMV capsid protein with an oil-in-water nanoemulsion and changing the pH and NaCl ionic strength, I, of the buffer through dialysis, causing the protein to assemble on the droplet surfaces (see FIG. 1). Transmission electron microscopy (TEM) of negatively stained VLDs reveals the presence of both ordered protein structures, including ring-like capsomers, on the surfaces of individual droplets. To probe a diversity of structures, we have encapsidated SDS-coated nanodroplets at five different buffer conditions, corresponding to the known phase behavior of the protein (Adolph, K. W.; Butler, P. J., Studies on the Assembly of a Spherical Plant Virus. I. States of Aggregation of the Isolated Protein. J. Mol. Biol. 1974, 88, 327-341; Adolph, K. W.; Butler, P. J., Assembly of a Spherical Plant Virus. Philos. Trans. R. Soc. Lond. B 1976, 276, 113-122; Bancroft, J. B.; Hills, G. J.; Markham, R., A Study of the Self-Assembly Process in a Small Spherical Virus. Formation of Organized Structures from Protein Subunits in Vitro. Virology 1967, 31, 354-379): ‘RNA-reassembly’ (pH=7.2, I=0.1 M), ‘hexagonal sheet’ (pH=6.2, I=0.1 M), ‘dimer’ (pH=6.2, I=1.0 M), ‘multi-shell’ (pH=4.8, I=0.1 M), and ‘empty shell’ (pH=4.8, I=1.0 M). We show TEM images of negatively stained VLDs for these buffers in FIG. 2a. Protein-coated nanodroplets can be distinguished from empty capsid shells because the uranyl acetate staining does not penetrate into the core of the coated droplets, so they appear noticeably brighter in the center. A darker ring around the edges of the droplets exists due to the trapping of the stain as the water contact line recedes during the evaporation process. This staining and drying process yields TEM images that provide excellent views of only one-half of the surface of each droplet. Because the images do not contain a significant signal from the protein on the other half, it is possible to identify and interpret the protein surface structure on individual droplets, rather than having to rely on reconstruction methods that presume an ordered structure.

For all five buffers, CCMV protein encapsidates nanodroplets, regardless of their size (FIG. 2a). Dimer buffer and RNA-reassembly buffer create VLDs efficiently without any loss of protein into empty shells. For the multi-shell buffer, we observe nanodroplets coated with single-, double-(dominant), and triple-shells (FIG. 2b). For the empty- and multi-shell buffer conditions, due to the slight excess of protein beyond what is required to coat the droplets, we observe encapsidated droplets and also empty shells.

To confirm that the protein is not simply deposited on the droplet surfaces during drying but actually assembles around the droplets while in solution, we have examined fluorescein isothiocyanate (FITC)-labeled CCMV capsid protein on microscale silicone oil droplets after dialyzing with RNA-assembly buffer. Strong fluorescence emanates from the surfaces of the droplets (FIG. 2a inset), indicating that they are coated with the labeled protein. By contrast, when droplets coated with cationic cetyl-trimethylammonium bromide (CTAB) surfactant are mixed with FITC-labeled CCMV proteins and dialyzed in the same manner, no surface fluorescence is observed, indicating that cationic surfactants are typically not suitable for creating protein-modified droplets with this particular protein.

We have also examined the structures and relative degree of order and disorder of protein on nanodroplets having different curvatures for RNA-reassembly conditions (FIG. 3). To enhance the images of the structures on the surfaces of individual VLDs, we remove the background and then reduce high-frequency noise using Fourier filtering. We identify complete capsomers as white rings that have an internal dark central spot and also a dark external trough surrounding the bright ring. The brighter regions indicate a higher density of proteins that project outward from the surfaces and the darker regions generally indicate a lower density of protein where stain becomes more highly concentrated in the local depressions. As the droplets become progressively larger than CCMV, complete capsomers become less prevalent and several other protein structures are observed on the less curved incompressible surfaces. In particular, imperfect capsomers, linear scar-like defects, and hexagonal web-like structures are seen, in sharp contrast to perfect icosahedral order on wild-type CCMV. Based on energy minimization, a greater relative coverage of the droplets by ordered capsomers might be expected when droplet sizes correspond to allowed integral triangulation numbers T (Bruinsma, R. F.; Gelbart, W. M.; Reguera, D.; Rudnick, J.; Zandi, R., Viral Self-Assembly as a Thermodynamic Process. Phys. Rev. Lett. 2003, 90, Art. No. 248101 pp. 1-4; Zandi, R.; Reguera, D.; Bruinsma, R. F.; Gelbart, W. M.; Rudnick, J., Origin of Icosahedral Symmetry in Viruses. Proc. Nat. Acad. Sci. 2004, 101, 15556-15560) (see FIG. 3-lower scale), yet this assumes that the structure and size of the capsomers will not be influenced by the underlying curvature and degree of compressibility of the core. Although our experiments at all buffer conditions do not reveal a higher degree of capsomer order on droplets that correspond to allowed T, this might occur at different pH and I than we have yet explored.

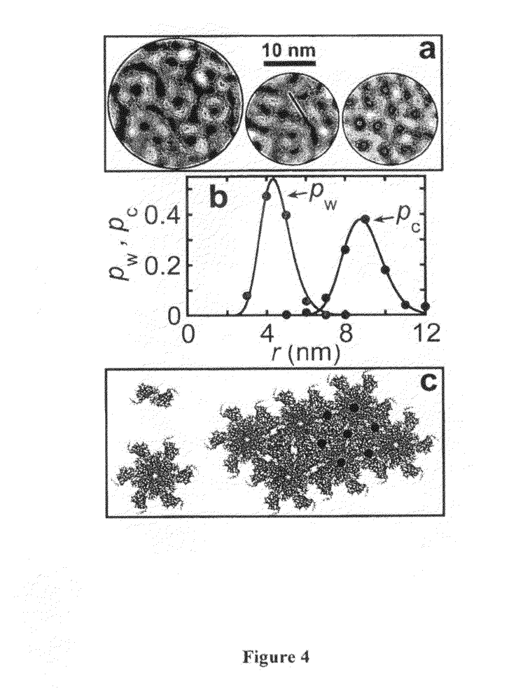

For smaller droplets closer to the size of the native virus, we have identified local hexagonal packing of capsomers (FIG. 4a-left), as can be seen on the native virus. Although we find numerous examples of six capsomers surrounding a central capsomer, five-fold coordinated capsomers without defects have not been observed on droplets significantly larger than CCMV. The distribution of center-to-center distances between neighboring six-fold capsomers is shown in FIG. 4b, and the average distance of 9.5 nm is in excellent agreement with that known from native CCMV (Speir, J. A.; Munshi, S.; Wang, G.; Baker, T. S.; Johnson, J. E., Structures of the Native and Swollen Forms of Cowpea Chlorotic Mottle Virus Determined by X-Ray Crystallography and Cryo-Electron Microscopy. Structure 1995, 3, 63-78).

On a number of larger droplets, we observe a hexagonal web-like structure of protein: regions of dark dots surrounded by interconnected white boundaries, or ‘web’ (FIG. 4a-right). Dark outer troughs characteristic of capsomers are absent. Although this protein web usually has local six-fold hexagonal order, in general, it can be disordered due to defects. The average distance between nearest-neighbor dots in the web is only 4.7 nm, about half the distance between the centers of capsomers (FIG. 4b). This is consistent with capsid protein self-assembling in a different manner on flatter, incompressible surfaces than on more highly curved, compressible surfaces (Bancroft, J. B.; Hills, G. J.; Markham, R., A Study of the Self-Assembly Process in a Small Spherical Virus. Formation of Organized Structures from Protein Subunits in Vitro. Virology 1967, 31, 354-379).

We propose that the mechanism of the formation of the hexagonal web structure of protein can be understood by considering the underlying symmetry and dense packing of self-assembled protein sub-units on incompressible surfaces of lower curvature. CCMV capsid protein is known to self-assemble into hexagonal capsomers of hand-in-glove dimmers (Tang, J.; Johnson, J. M.; Dryden, K. A.; Young, M. J.; Zlotnick, A.; Johnson, J. E., The Role of Subunit Hinges and Molecular ‘Switches’ in the Control of Viral Capsid Polymorphism. J. Struct. Biol. 2006, 154, 59-67; Adolph, K. W.; Butler, P. J., Studies on the Assembly of a Spherical Plant Virus. I. States of Aggregation of the Isolated Protein. J. Mol. Biol. 1974, 88, 327-341) that have been identified by x-ray cystallography. These dimers are energetically favored over monomers in many buffer conditions; a protruding arm of one protein is inserted into the folded region of its partner and is held by an attraction, and vice-versa. Six hand-in-glove dimers can come together to form a capsomer that has six protruding arms in a structure resembling a gear (FIG. 4c-lower left). Such capsomer structures are also energetically favored over a random assembly of dimers. When these gear-like hexagonal capsomers of dimers self-assemble and then densely pack to cover a flat surface, they can create hexagonal arrays of capsomers that have regions that are depleted of protein at half of the center-to-center distance between neighboring capsomers. This packed-gear structure would give the appearance of the web that we observe: a hexagonal array of dark spots where the protein density is lower and an interconnected hexagonal network of bright web where the protein density is higher. For self-assembly of protein on a flat surface, it is reasonable to assume that the folded capsid protein and dimers exist in only a single conformation and do not distort into the three known conformations that are required for assembling five-fold coordinated capsomers on core structures that have higher curvature comparable to that of the wild-type virus.

In addition to the ordered web, we also observe elongated protein scars that are dark troughs surrounded by white rims (FIG. 4a-middle). These protein scars bear some resemblance to scar defects found in the packing of monodisperse solid spheres on the surfaces of curved liquid droplets (Bausch, A. R.; Bowick, M. J.; Cacciuto, A.; Dinsmore, A. D.; Hsu, M. F.; Nelson, D. R.; Nikolaides, M. G.; Travesset, A.; Weitz, D. A., Grain Boundary Scars and Spherical Crystallography. Science 2003, 299, 1716-1718; Bowick, M.; Cacciuto, A.; Nelson, D. R.; Travesset, A., Crystalline Order on a Sphere and the Generalized Thomson Problem. Phys. Rev. Lett. 2002, 89, Art. No. 185502 pp. 1-4), a controlled variety of ‘Pickering emulsions’ (Tarimala, S.; Dai, L. L., Structure of Microparticles in Solid-Stabilized Emulsions. Langmuir 2004, 20, 3492-3494; Pickering, S. U., Emulsions. J. Chem. Soc. Trans. Lond. 1907, 91, 2001-2021; Subramaniam, A. B.; Abkarian, M.; Stone, H. A., Controlled Assembly of Jammed Colloidal Shells on Fluid Droplets. Nat. Mater. 2005, 4, 553-556), yet capsid protein scars are distinctly different. Protein scars do not consist simply of line defects between fully formed hexagonal ring-like capsomers on incommensurately sized droplets; instead they indicate disorder of the protein at a smaller scale than even the capsomer unit itself. Several mechanisms combine to produce the scar defects: incommensurate sizes of the droplets relative to those corresponding to allowed T-numbers for icosahedral structures, and surface jamming of the protein that strongly inhibits protein reorientation and rearrangement once the surface is completely covered.

### Discussion of Results

A variety of protein structures, including dimers, partial capsomers, and complete capsomers, may become jammed into locally disordered states (Liu, A. J.; Nagel, S. R., Jamming Is Not Just Cool Any More. Nature 1998, 396, 21-22) on incompressible spherical surfaces that have reduced curvature in a manner reminiscent of out-of-equilibrium glasses and gels. Additional defects may arise because protein adsorbed at high densities may not be able to change conformation and reorganize into lowest-energy ordered states, as when forming around RNA. Controlling the relative protein coverage and examining the kinetics of the process of encapsidation will provide greater insight into how ordered and disordered protein structures arise on the surfaces of VLDs. By adjusting the pH and ionic strength, it may be possible to encapsidate droplets, nanoparticles, and synthetic polymers with a controlled number of capsid shells.

By interpreting TEM images of individual encapsidated nanodroplets, we have revealed a range of new structures, including defected capsomers, hexagonal web, and scars, on the surfaces of the droplets. The discovery of these structures provides significant new insight into the nature of protein conformations on curved surfaces. Moreover, it shows that non-equilibrium protein structures can exist on encapsidated nanoscale objects due to surface jamming on an incompressible charged template and that the picture of thermodynamic self-assembly of perfect icosahedral shells may be correct only in certain limiting cases.

Proteins useful for making protein-modified droplets may be obtained from viruses that are members of the following families of viruses: Adenoviridae, Anellovirus, Arenaviridae, Arteriviridae, Ascoviridae, Asfarviridae, Astroviridae, Asunviroidae, Baculoviridae, Barnaviridae, Benyvirus, Birnaviridae, Bornaviriclae, Bromoviridae, Bunyaviridae, Caliciviridae, Caulimoviridae, Cheravirus, Chrysoviridae, Circoviridae, Closteroviridae, Comoviridae, Coronaviridae, Corticoviridae, Cystoviridae, Deltavirus, Dicistroviridae, Endornavirus, Filoviridae, Flaviviridae, Flexiviridae, Furovirus, Fuselloviridae, Geminiviridae, Guttaviridae, Hepadnaviridae, Hepeviridae, Herpesviridae, Hordeivirus, Hypoviridae, Iflavirus, Inoviridae, Iridoviridae, Leviviridae, Lipothrixviridae, Luteoviridae, Marnaviridae, Metaviridae, Microviridae, Mimivirus, Myoviridae, Nanoviridae, Narnaviridae, Nimaviridae, Nodaviridae, Ophiovirus, Orthomyxoviridae, Ourmiavirus, Papillomaviridae, Paramyxoviridae, Partitiviridae, Parvoviridae, Pecluvirus, Phycodnaviridae, Picornaviridae, Plasma viridae, Podoviridae, Polydnaviridae, Polyomaviridae, Pospiviroidae, Potyviridae, Poxviridae, Pseudoviridae, Reoviridae, Retroviridae, Rhabdoviridae, Rhizidiovirus, Roniviriciae, Rudiviridae, Sadwavirus, Salterprovirus, Sequiviridae, Siphoviridae, Sobemovirus, Tectiviridae, Tenuivirus, Tetraviridae, Tobamovirus, Tobravirus, Togaviridae, Tornbusviridae, Totiviridae, Tymoviridae, Umbravirus, and Varicosavirus. Proteins useful for making protein-modified droplets may also be obtained from members of other families of viruses not in this list and also from families of viruses yet to be discovered and studied.

In addition to proteins from viruses, proteins taken from bacteria, fungi, plants, animals, and sponges can be used to make protein-modified droplets if such proteins can be effectively isolated, separated, and manipulated in a manner that brings them into proximity with the surfaces of droplets in a manner that is created by an attractive interaction of the protein with the droplet surface.

Proteins useful for making protein-modified droplets may have a variety of functions, including but not limited to: structural protein, non-structural protein, coat protein, capsid protein, core protein, envelope protein, matrix protein, transmembrane protein, membrane associated protein, non-structural protein, nucleocapsid protein, filamentous protein, capping protein, crosslinking protein, glycoprotein, and motor protein.

The examples of protein-modified droplets we have provided are of a single capsid protein purified from Cowpea Chlorotic Mottle Virus (CCMV), a member of the family of plant viruses, Bromoviridae.

Viruses can have more than one type of capsid protein. In the particular examples we have shown, we have substituted a polyanionic droplet in place of polyanionic genetic material as a template for protein assembly, a wide variety of viral capsid proteins can be effectively attracted to the surface of the charged droplet. Thus, for viruses having two or more capsid proteins, an appropriate stoichiometric ratio of different protein types would certainly provide sufficient structural features to modify and/or enclose the droplets. Moreover, for viruses that naturally produce two or more types of capsid proteins, even a single type of capsid protein that has been purified is sufficient to modify and/or enclose the droplets; having all different types of capsid proteins present from a particular virus is not necessary. The main requirement is that the charge on the surface of the droplet, the pH of the solution, and the ionic composition and ionic strength of the solution must be adjusted such that the protein experiences an attractive interaction with the droplet surface and thereafter remains proximate to said droplet surface.

Once the protein coating has been formed around the droplet it can be advantageous in certain applications to also form a lipid coating, a lipoprotein coating, or a lipid-protein coating that surrounds the protein layer, thereby creating a total structure resembling an enveloped virus. Thus, this structure contains an inner droplet core, a surface active agent typically adsorbed to the core, a layer of protein surrounding the droplet core with surface active agent, and a layer of lipid, lipoprotein, or lipid-protein.

The selective uptake and localization of specific viruses, both with and without the enveloped layer, by specific tissues and organs in higher-level organisms is well known and is discussed in a book such as “Basic Virology”, 2nd edition, by E. K. Wagner and M. J. Hewlett, Blackwell Publishing (2004). Protein-modified droplets present the same protein structures to biological organisms as do naturally occurring virions that contain genetic material, so the preferential uptake and localization of protein-modified droplets will occur in the same tissues and organs as is found for natural virions that display the same proteins.

The current invention is not limited to the specific embodiments of the invention illustrated herein by way of example, but is defined by the claims. One of ordinary skill in the art would recognize that various modifications and alternatives to the examples discussed herein are possible without departing from the scope and general concepts of this invention.

We claim:

1. A protein-modified droplet, comprising:
a droplet comprising a liquid material; and
a protein structure formed to at least partially enclose said droplet,
wherein said protein structure comprises a plurality of protein molecules having an affinity to at least a region of said droplet during formation of said protein structure, and
wherein said droplet has a maximum dimension of at least about 1 nm and less than about 1000 nm.

2. A protein-modified droplet according to claim 1, wherein said droplet has a maximum dimension of at least about 5 nm and less than about 100 nm.

3. A protein-modified droplet according to claim 1, wherein said protein structure substantially surrounds said core of a first liquid material.

4. A protein-modified droplet according to claim 1, wherein said liquid material of said droplet comprises a hydrophobic material.

5. A protein-modified droplet according to claim 1, wherein said liquid material of said droplet comprises at least one material selected from the group of materials consisting of an oil, a silicone oil, a hydrocarbon oil, a petroleum oil, a fuel oil, a wax, a fat, a fluorinated oil, a non-volatile oil, a volatile oil, an aromatic oil, an oil derived from a plant material, an oil derived from an animal material, an oil derived from a natural source, a distilled oil, an extracted oil, a cooking oil, a food oil, a lubricant, a reactive material that is predominantly hydrocarbon in composition, an epoxy material, an adhesive material, a polymerizable material, a thermotropic liquid crystal, a lyotropic liquid crystal, an acidic oil, a basic oil, a neutral oil, a natural oil, a polymer oil, and a synthetic oil.

6. A protein-modified droplet according to claim 5, wherein said liquid material of said droplet further comprises a biologically active agent that is dispersible in said at least one material.

7. A protein-modified droplet according to claim 6, wherein said biologically active agent is selected from the group of materials consisting of drug molecules, anti-cancer molecules, therapeutic molecules, hormone molecules, agonist molecules, antagonist molecules, inhibitor molecules, suppressor molecules, sensitizer molecules, antidepressant molecules, antiviral molecules, antifungal molecules, antibacterial molecules, bioavailability enhancer molecules, RNA-binding molecules, DNA-binding molecules, toxin molecules, dye molecules, fluorescent molecules, biomolecules, nutrients, vitamins, flavors, enzymes, radioactive isotopes, non-radioactive isotopes, nanoparticles, and imaging contrast enhancement agents.

8. A protein-modified droplet according to claim 3, wherein said protein structure is a monolayer of protein molecules.

9. A protein-modified droplet according to claim 3, wherein said plurality of protein molecules form an at least a partially ordered protein structure.

10. A protein-modified droplet according to claim 9, wherein said plurality of protein molecules contains at least one assembled protein substructure from the group of substructures consisting of protein dimers, trimers, tetramers, pentamers, hexamers, heptamers, octamers, pentons, hexons, fibers, web-like structures, and capsomers.

11. A protein-modified droplet according to claim 3, wherein said protein structure is a plurality of protein layers.

12. A protein-modified droplet according to claim 1, wherein said plurality of protein molecules that form said protein structure is a plurality of naturally occurring protein molecules.

13. A protein-modified droplet according to claim 12, wherein said protein structure is formed from virus capsid proteins that are known to preferentially enter and concentrate within at least one of specific types of sub-cellular structures, specific types of cells, specific biological tissues, and specific biological organs.

14. A protein-modified droplet according to claim 12, wherein said naturally occurring plurality of protein molecules is a plurality of viral capsid protein molecules.

15. A protein-modified droplet according to claim 1, wherein said plurality of protein molecules that form said protein structure is a plurality of synthetic polypeptide molecules.

16. A protein-modified droplet according to claim 1, wherein said droplet comprises a hydrophobic material in a composition thereof.

17. A protein-modified droplet according to claim 1, wherein said droplet comprises amphiphilic surface active molecules adsorbed on said liquid material, said amphiphilic surface active molecules having charges suitable to attract said plurality of protein molecules.

18. A protein-modified droplet according to claim 17, wherein said amphiphilic surface active molecules are anionic surfactant molecules.

19. A protein-modified droplet according to claim 1, further comprising at least one of a lipid molecule, a lipo-protein molecule, a membrane protein, and an antigen attached to said protein structure.

20. A protein-modified droplet according to claim 3, further comprising a lipid membrane formed on said protein structure.

21. A protein-modified droplet according to claim 1, wherein said plurality of protein molecules that form said protein structure comprises a plurality of different types of protein molecules.

22. A composition comprising a plurality of protein-modified droplets dispersed in an aqueous solution, wherein each said protein-modified droplet comprises:
a droplet comprising a liquid material; and
a protein structure formed to at least partially enclose said droplet,
wherein said protein structure comprises a plurality of protein molecules having an affinity to at least a region of said droplet during formation of said protein structure, and
wherein said droplet has a maximum dimension of at least about 1 nm and less than about 100 nm.

23. A method of producing protein-modified droplets, comprising:
supplying first and second immiscible liquid materials;
adding a stabilizing agent to at least one of said first and second immiscible liquid materials;
emulsifying said first and second liquid materials to form a plurality of droplets of said second liquid material in said first liquid material that are stabilized by said stabilizing agent, each droplet of said plurality of droplets having a maximum dimension of at least about 1 nm and less than about 100 nm;
adding protein molecules at least one of prior to and after said emulsifying; and allowing a protein structure to form to at least partially enclose each of said plurality of droplets,
wherein said stabilizing agent and said protein molecules added are of types that have mutual electrostatic attractions to each other when said stabilizing agent is attached to said droplets.

24. A method of producing protein-modified droplets according to claim 23, wherein each said protein structure is an exostructure that lies outside of a corresponding droplet.

25. A method of producing protein-modified droplets according to claim 23, further comprising evaporating said first liquid material in which said plurality of protein modified droplets have been formed, wherein said protein structure inhibits coalescence of said protein-modified droplets.

26. A method of producing protein-modified droplets according to 23, further comprising causing protein molecules to aggregate in said first liquid material comprising at least one of dialysis, titration, mixing, changing ionic concentrations in said first liquid material, changing pH of said first liquid material, changing buffer type of said first liquid material, and causing a chemical reaction in said first liquid material.
